(19) United States
(12) Patent Application Publication
(10) Pub. No.: US 2008/0125663 A1
(43) Pub. Date: May 29, 2008

(54) Pre-And Intra-Operative Imaging of Testicular Torsion

(75) Inventor: Drajan Golijanin, Rochester, NY (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: University of Rochester Medical Center, Rochester, NY (US)

(21) Appl. No.: 11/780,430

(22) Filed: Jul. 19, 2007

Related U.S. Application Data
(60) Provisional application No. 60/832,542, filed on Jul. 20, 2006.

(51) Int. Cl. A61B 5/0275; A61B 17/28

(52) U.S. Cl. 600/479; 600/312; 606/205

(57) ABSTRACT

The invention provides methods for visualizing perfusion or lack thereof in the spermatic cord and testicle, as well as for detecting testicular trauma. A surgical forceps adapted to facilitate such visualization is also provided.

### CROSS-REFERENCES TO RELATED APPLICATIONS

This application claims the benefit of U.S. Provisional Application No. 60/832,542, filed Jul. 20, 2006, the contents of which are hereby incorporated by reference.

### STATEMENT AS TO RIGHTS TO INVENTIONS MADE UNDER FEDERALLY SPONSORED RESEARCH AND DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

Not Applicable

### BACKGROUND OF THE INVENTION

In some males, anatomic abnormalities, such as high attachment to the tunica vaginalis (“bell clapper deformity”), allow the testicle to rotate. If the testicle rotates sufficiently, the testicular artery and vein are occluded, resulting in hypoperfusion or absence of perfusion in the testicle. If this loss of blood supply is not corrected within 6 to 12 hours, it results in the death of the testicle. This rotation, known as testicular torsion, is a urologic emergency. Testicular torsion can occur in neonates, but is most common in males who are between 12 to 15 years of age.

A variety of medical techniques for imaging biological tissues and organs are known. These include traditional x-rays, ultra-sound, magnetic resonance imaging (MRI), and computerized tomography (CT). Techniques such as MRI, micro-CT, micro-positron emission tomography (PET), and single photon emission computed tomography (SPECT) have been explored for imaging function and processes in small animals or in vivo, intra operatively. These technologies offer deep tissue penetration and high spatial resolution, but are costly and time consuming to implement. In the case of a patient presenting with sudden scrotal pain, pulsed or Doppler ultrasound may be used to differentiate testicular torsion from other possible causes. These method require an experienced ultrasonographer and special equipment, and physical contact of the ultrasound equipment against the already painful scrotum.

It would be desirable to have a less cumbersome technique that can be performed by members of the emergency department or surgical team and which can increase the opportunity to visualize testicular perfusion without the need to contact physically an already painful scrotum. The present invention fills these and other needs.

### BRIEF SUMMARY OF THE INVENTION

The invention provides methods for determining vascular perfusion of the testicles and related structures, and a forceps designed to facilitate such determinations.

In some embodiments, the invention provides methods of diagnosing occlusion of blood supply through a spermatic cord to a testicle in a subject, comprising: (a) administering systemically to said subject a dye which fluoresces at an emission wavelength when said dye is contacted with an excitation wavelength; (b) exposing (i) the spermatic cord, (ii) the testicle, or (iii) both the spermatic cord and the testicle, to a source of illumination comprising the excitation wavelength under conditions such that any fluorescent dye in the spermatic cord, testicle, or both the spermatic cord and testicle fluoresces; (c) detecting the presence or absence of fluorescence of said dye in the spermatic cord, said testicle, or in both said spermatic cord and said testicle, wherein the presence of fluorescence throughout the spermatic cord, or in the testicle, or both, indicates a lack of occlusion of blood supply to said testicle and the absence of fluorescence throughout the spermatic cord, or in the testicle, or both, indicates an occlusion of blood supply to said testicle. In some embodiments, the exposing of step (b) is of the testicle. In some embodiments, the exposing of step (b) is of the spermatic cord. In some embodiments, the dye is administered intravenously. In some embodiments, the presence or absence of said fluorescence of said spermatic cord or said testicle is visualized on a image display. In some embodiments, the source of illumination is a laparoscopic instrument. In some embodiments, the dye is a near infrared dye. In some embodiments, the dye is a tricarbocyanine dye or an analog thereof. In some embodiments, the tricarbocyanine dye is indocyanine green. In some embodiments, the subject is a human. In some embodiments, the source of illumination exposes said spermatic cord or said testicle or both to the light through the scrotum. In some embodiments, the detection of the presence or absence of fluorescence is made through the scrotum. In some embodiments, the diagnosis is made during a surgical operation. In some embodiments, the dye is administered within 2 hours of said exposure to the source of illumination. In some embodiments, the dye is administered within 1 hour of the exposure to the source of illumination. In some embodiments, the dye is administered and the spermatic cord or the testicle is exposed within 5 minutes to the source of illumination. In some embodiments, the dye is administered between 5 minutes and 1 hour before the surgical operation.

In a further group of embodiments, the invention provides methods for determining the location of an undescended testicle in a neonate, comprising: (a) administering systemically to a neonate with an undescended testicle a dye which fluoresces at an emission wavelength when the dye is contacted with an excitation wavelength; (b) exposing the lower abdomen of the neonate to a source of illumination comprising the excitation wavelength under conditions such that fluorescent dye in the undescended testicle fluoresces; and (c) detecting the presence of fluorescence of the dye in the testicle, thereby localizing the testicle. In some embodiments, the dye is a near infrared dye. In some embodiments, the dye is a tricarbocyanine dye or an analog thereof. In some embodiments, the dye is indocyanine green. In some embodiments, the exposing of step (b) is from outside the abdomen. In some embodiments, the exposing of step (b) is from an intra-abdominal laparoscopic instrument.

In still a further group of embodiments, the invention provides methods of determining perfusion of a testicle in a subject following orchidopexy, comprising: (a) administering systemically to the subject a dye which fluoresces at an emission wavelength when the dye is contacted with an excitation wavelength; (b) exposing the testicle to a source of illumination comprising the excitation wavelength under conditions such that fluorescent dye in said testicle fluoresces; (c) observing the presence or absence of fluorescence of the dye in the testicle, wherein the presence of fluorescence indicates perfusion of the testicle and the absence of fluorescence indicates its absence, thereby determining perfusion of the testicle. In some embodiments, the dye is a near infrared dye. In some embodiments, the dye is a tricarbocyanine dye or an analog thereof. In some embodiments, the dye is indocyanine green.

In yet a further group of embodiments, the invention provides methods for determining if a subject has suffered testicular trauma, comprising: (a) administering systemically to the subject a dye which fluoresces at an emission wavelength when the dye is contacted with an excitation wavelength; (b) exposing the testicle to a source of illumination comprising the excitation wavelength under conditions such that fluorescent dye in said testicle fluoresces; (c) observing whether the dye is present only in the testicle or is extravasated from the testicle, wherein the presence of the extravasation indicates the presence of testicular trauma and the absence of the extravasation indicates the absence of testicular trauma. In some embodiments, the dye is a near infrared dye. In some embodiments, the dye is a tricarbocyanine dye or an analog thereof. In some embodiments, the dye is indocyanine green. In some embodiments, the exposing of step (b) is from outside the scrotum. In some embodiments, the exposing or said detecting, or both, is conducted using a laparoscopic instrument or instruments.

In still another group of embodiments, the invention provides surgical forceps for facilitating observing fluorescence in a subject organ. The forceps comprise a first and a second elongate arm pivoted in a scissors fashion, first and second finger grips at the proximal ends of each of the first and second arms, respectively, and first and second jaws at the ends of each of the first and second arms distal from said finger grips, which first and second jaws have an inward aspect and an outside aspect, wherein at least the first jaw has a shield opaque to fluorescent light of a selected spectrum attached to the outward aspect of the jaw. In some embodiments, the spectrum is the near infrared spectrum. In some embodiments, the second jaw also has a shield opaque to fluorescent light of the selected spectrum attached to the outward aspect of the jaw. In some embodiments, the shield is oval in shape except along an edge attached to the jaw. In some embodiments, the shield is semi-circular in shape except along an edge attached to the jaw. In some embodiments, the jaws do not come into contact when the arms are closed. In some embodiments, the arms further comprise a ratcheting mechanism proximal to said finger grips which, when engaged, holds the arms in a closed position. In some embodiments, the jaws are padded.

### BRIEF DESCRIPTION OF THE DRAWINGS

FIG. 1. FIG. 1 shows a surgical forceps adapted to facilitate visualization of fluorescence in an organ, such as the testicle or spermatic cord. The forceps have finger rings 10, arms 20, jaws 30, and one or more shields 40 opaque to near-infrared light attached to the outside aspect of the jaws.

FIG. 2. FIG. 2 shows the testes of a rat after unilateral interruption of blood flow to the right testis. Obstruction was with a vascular clamp. Indocyanine green was administered intravenously. The rat displayed obvious differential perfusion after occlusion.

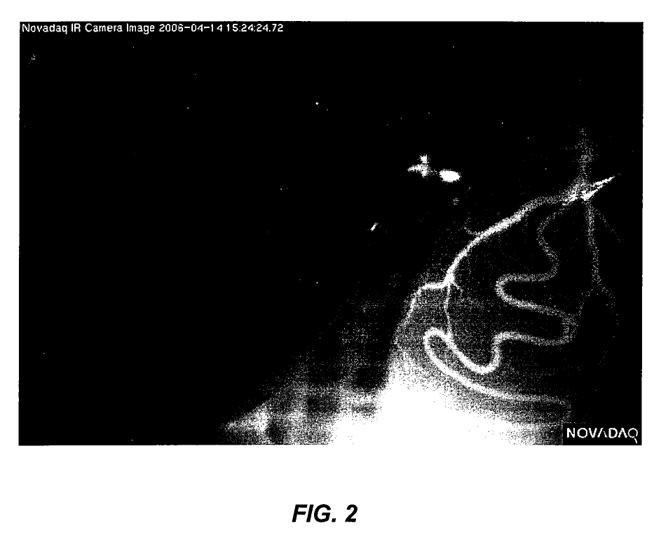

FIGS. 3A-F. FIGS. 3A to F show the testes of a second rat after unilateral interruption of blood flow to the right testis. Obstruction was with a bull dog clamp. Indocyanine green (ICG) was administered intravenously. FIG. 3A: The testes show no fluorescence prior to administration of ICG. FIG. 3B: Bull dog clamp vascular obstruction of the right spermatic cord resulted in absence of fluorescence in the right testicle following intravenous administration of 0.5 ml of 2.5 mg/ml ICG. FIG. 3C: Reinsertion of the testicles into the scrotum resulted in low fluorescence of the right hemiscrotum. FIG. 3D: Reinsertion of the testicles into the scrotum resulted in intense fluorescence of the left hemiscrotum. Minimal fluorescence of the right side of the scrotum was due to the presence of ICG in subcutaneous and cutaneous vessels of the scrotum. Images in FIGS. 3C and D were captured during manipulation of the scrotum to bring the testicles closer to the scrotal wall and o minimize the transfer of fluorescence between the two sides of the scrotum. FIG. 3E: Image collected at the tip of the scrotum without manipulation. FIG. 3F: Removal of the clamp from the spermatic cord resulted in restoration of blood flow to the right testicle, as indicated by restoration of fluorescence of the testicle.

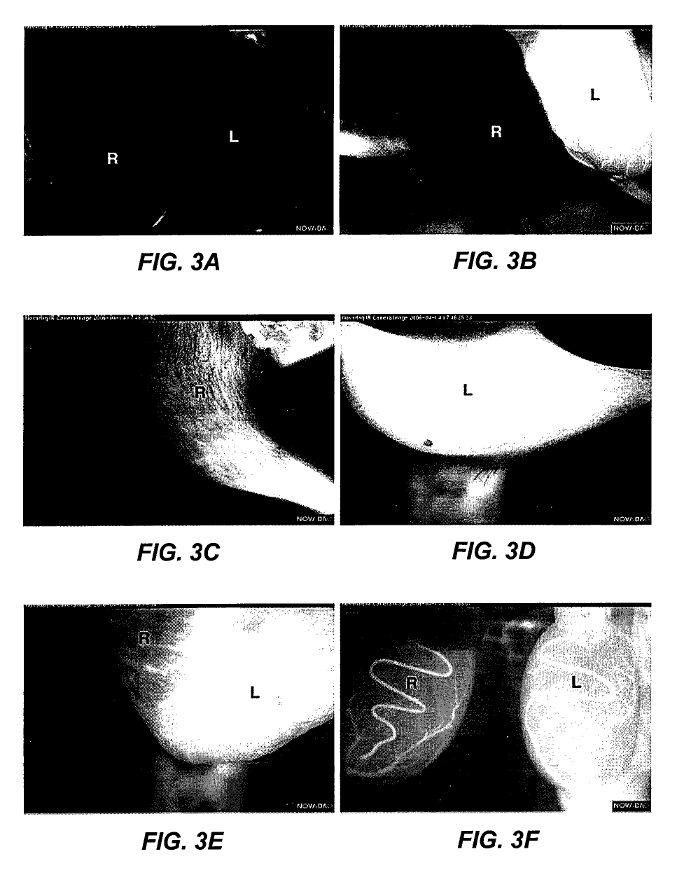

### DETAILED DESCRIPTION OF THE INVENTION

### Introduction

Testicular torsion is a urologic emergency. The patient usually presents with a sudden onset of scrotal pain, often accompanied by swelling, nausea and abdominal pain. Torsion is diagnosed by symptoms and by palpation of the testicle. Treatment usually involves placing the patient under anesthesia, opening the scrotum, untwisting the spermatic cord, and suturing the involved testicle to the interior of the scrotum to prevent recurrence. Since testicular torsion often results from an anatomic predisposition, the uninvolved testicle is usually also suture fixated, typically to the tunica dartos, to prevent future torsion.

Time is of the essence in diagnosis and treatment. The rate for salvaging the affected testicle in males who are treated within six hours of the onset of pain is estimated to be 80 to 100%, but drops sharply after more than six to eight hours. The salvage rate drops to zero after twelve hours. Accordingly, timely and correct diagnosis is critical to save the patient's testicle.

Pulsed or Doppler ultrasound may be used to differentiate testicular torsion from other causes of pain. Typically, ultrasound of testicular torsion shows enlargement of the involved testicle. Doppler ultrasound can reveal diminished or no blood flow to the testicle. Unfortunately, ultrasound imaging usually requires the presence of an experienced sonographer, who may not be readily available, for example, in the middle of the night, particularly in smaller facilities. Given the short window of time for diagnosis and treatment, it would be desirable to avoid the time which may be required for an experienced ultrasonographer to be contacted and to arrive at the patient's side. Moreover, Doppler sonography does not permit correct determination of arterial blood flow in the testicular area, especially in small children. Even experienced sonographers can mistake venous blood flow for arterial blood flow in such patients.

The present invention solves these problems and provides a more direct diagnosis of the problem. In the methods of the present invention, a non-toxic fluorescent dye is administered to the patient. Conveniently, the dye is administered as an intravenous solution. The dye circulates through the patient's systemic circulation. Such dyes typically have a known excitation frequency and a known emitting frequency. A light source capable of emitting light of the dye's excitation frequency is positioned in proximity to the scrotum so as to permit light from the light source to illuminate the affected testicle, or spermatic cord, or both, in a sufficient amount to excite the dye. A camera or other device capable of capturing an image of light received at the emission frequency of the dye is positioned to receive light emitted from dye in the testicle or spermatic cord. Conveniently, the light goes through a filter capable of selectively passing light in the dye's emission frequency while blocking light at the dye's excitation frequency, thus permitting the receiving device to provide an image based on emission light from the dye.

In some embodiments, the dye used is indocyanine green, or “ICG.” After intravenous injection, ICG is bound within 1 to 2 seconds, mainly to globulins (1-lipoproteins), and remains intravascular, with normal vascular permeability. ICG is not metabolized in the body and is excreted exclusively by the liver, with a plasma half-life of 3 to 4 minutes. Thus, ICG is available in the vasculature almost immediately after injection for visualization of vasculature in the scrotal area in general and blood supply to the testicles in particular.

The prompt and accurate diagnosis of testicular torsion allows immediate surgical intervention. The methods of the invention also allow the intraoperative evaluation of the success of the detorsion (untwisting of the spermatic cord) by evaluating the reperfusion of the cord and testicle. If desired, the practitioner performing the procedure can attempt manual detorsion without surgically entering the scrotum, and evaluate whether the attempt is successful before commencing the surgical procedure.

The methods of the invention can also be used for other procedures involving the testicles and vascular perfusion. For example, the methods can be used for preoperative location of undescended testicles, particularly in cases and evaluations of neonates in which gender is unclear. The methods can also be used in intraoperative laparoscopic localization of intra-abdominal testicles, allowing identification of the testicular blood vessels, presence of the testicle, and visualization assistance during, for example, the first of the Fowler-Stephens two step procedure for fixing undescended testicles into the scrotum in pediatric patients. (Performance of the Fowler-Stephens method as a complete laparoscopic procedure is discussed in, for example, Esposito and Garipoli, J Urol 158(5):1956 (1997)).

The methods of the invention further permit post-orchidopexy follow-up of testicular perfusion and size. Orchidopexy, such as the Fowler-Stephens procedure, is the surgical repair of undescended testicles. Currently, follow-up studies of testicular perfusion and size in such patients is performed by ultrasound. The methods of the invention permit direct visualization of the blood supply to the testicle as well as of size, and should replace ultrasound as the standard for follow-up studies.

In another set of uses, the methods of the invention allow evaluating whether injury to the tunica albuginea or the testes has occurred as a result of testicular trauma. Near-IR visualization permits rapid identification of extravasation of blood through the tunica albuginea or the testes following trauma to the scrotal area, as well as any vascular compromise. The presence of extravasation of blood indicates the presence of testicular rupture, or violation of the tunica albuginea of the testis, while the absence of extravasation indicates the absence of such trauma.

In each of these applications, the use of near-IR fluorescent dyes permits trans-scrotal imaging on a continuous basis, while physical contact with the scrotum can be avoided.

In some embodiments, visualization of the vasculature is facilitated by use of a surgical forceps designed to facilitate visualization of fluorescence in an organ of interest, and particularly in the testicle or spermatic cord. The forceps have at least one shield that is opaque to light of a selected fluorescence spectrum. That is, for use with dyes which fluoresce in infrared light, for example, the shield will be opaque to infrared light, while for use with dyes which fluoresce under ultraviolet light, the shield will be opaque to ultraviolet light. The shield may optionally be made from material that is opaque to light of any frequency so that it can be used with any fluorescent dye the practitioner chooses. Preferably, the shield is opaque to near-infrared light, so that it can be used with, for example, ICG. The shield is positioned between the testicles or the portions of the scrotum to facilitate visualization of fluorescence from a spermatic cord or testicle of interest by blocking fluorescence from areas not being visualized. For example, the forceps are particularly useful when intense fluorescence from one testicle would otherwise make it difficult to visualize or image weaker fluorescence from the other testicle, or when fluorescence from blood vessels in the scrotum would otherwise make it difficult to see or image fluorescence in the testicle or spermatic cord.

The forceps may be designed to lock the jaws in a closed position to facilitate holding the forceps in position. Forceps handles that permit the jaws to lock in position is well known in the art. For imaging through the scrotum, as in a pre-operative diagnosis of testicular torsion or trauma, the jaws of the forceps may be used to hold the forceps clamped to the scrotum with the shield positioned to block any fluorescence from the section of the scrotum or testicle whose image is not desired at that moment. Used intra-operatively, the forceps may be held manually in position within the scrotum or the jaws may be closed and locked on a portion of the scrotum to hold the forceps in position.

As those in the art will appreciate, surgical forceps are instruments typically comprising two arms connected at a pivot point disposed along the length of the two arms. Each arm has two ends, one to each side of the pivot point. Typically, at the first end, each of the arms have a handle or finger grip, such as a finger ring, intended to facilitate moving the arms together or apart at that end, while at the second end, each of the arms form a jaw for grasping or compressing items placed between the jaws when the handles or finger grips on the first end are brought together. The arms are typically straight, curved or arcuate. Typically, the forceps have a design facilitating locking the two arms in position when fully or partly closed to relieve the user from having to continually exert pressure once the forceps has been positioned and closed to the desired position. For example, each arm of the forceps may have a protrusion shaped to releasably hold the other when the arms are closed to a desired position. Conveniently, the forceps may also have a spring attached to the arms to facilitate opening the arms when the locking mechanism is released. A large variety of forceps are known and commercially available.

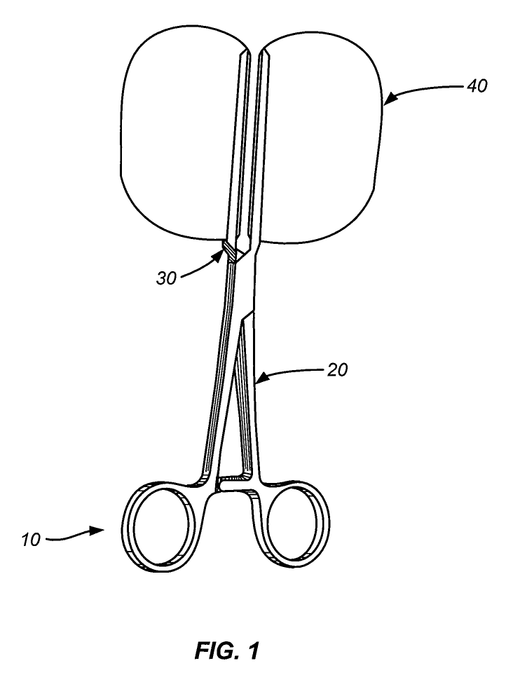

The inventive forceps has handles or finger grips, such as the finger rings typical of forceps, scissors, and other devices designed for manipulation in the hands. The arms can be straight or curved. For example, the arms may be arcuate or generally arcuate in shape. FIG. 1 shows an embodiment of the invention in which the forceps have finger rings, 10. The forceps has two arms 20, and jaws 30 typical of surgical forceps. Attached to the side of the jaws facing outward along a line parallel to the length of the forceps are one or two shields 40 opaque to near-infrared (“NIR” or “nearIR”) light. The inside aspect of the jaws can be serrated to facilitate gripping the scrotum, or can be padded to reduce pressure on scrotal skin.

The shields of the forceps will have a shape that is generally semi-circular, ovate or elongated along in the portion that extends beyond the jaws to block fluorescence. To facilitate attachment to the jaw, the shield may be straight along the edge where the shield is in contact with the jaw, to which it may be attached by, for example, adhesive, rivets, solder, mini-welds, screws, or other techniques known in the art. Alternatively, the jaw and the shield are not formed separately and then attached, but are made as a single unit. The jaws themselves are preferably padded, and preferably to do not close completely even when the handle is fully squeezed to avoid injury to the scrotal skin during use, as well as pain, while permitting a firm grip to be maintained on the scrotum during visualization and imaging. The jaws may be straight or curved, and may be grooved to enhance grip.

### Instrumentation

For convenience, the following discussion will refer to instrumentation optimized for use with the exemplar dye ICG. Persons of skill are, of course, aware of the excitation and emission frequencies of other fluorescent dyes and can adjust the device as needed for use with respect to other dyes as desired.

Conveniently, the device used for visualization of the testicle comprises both a laser and a camera, although the two may be provided separately. For use with ICG, the laser preferably consists of a laser diode providing a maximum of 3 W output at 806 nm. For other dyes, the laser diode is selected to provide a light with a wavelength at an excitation frequency appropriate for the dye selected. For convenience of reference, the discussion below generally refers to the exemplar dye ICG. Persons of skill will recognize that the other dyes mentioned herein as suitable for use in the inventive methods and procedures could be substituted for ICG, with the light source selected or adjusted to provide illumination optimized for the excitation frequency suitable for the particular dye chosen and the device for capturing the light emitted by the dye being selected or adjusted to be able to receive light of the appropriate frequency.

The laser output is decollimated (i.e. optics are used to spread out the laser light from a tight beam) to provide even illumination over a field of view, for example, 7.6 cm by 7.6 cm at a working distance of 30 cm. Light emitted from the dye is captured by a capture unit, which is typically a video camera containing a charge-coupled device (“CCD”) or a complementary symmetry metal oxide semiconductor (“CMOS”) image sensor capable of detecting fluorescence in the emission frequency of the chosen dye. For use with ICG, the image sensor should be sensitive into the near infrared spectrum and is preferably equipped with an 815 nm edge filter. In some embodiments, the laser or camera or both, are supported by an articulated arm connected to a wheeled base. This allows the imaging head to be moved into close proximity to the surgical table and for vertical movement of the head to attain the correct focal distance above the area of interest. The imaging head and extension arm that protrudes over the surgical field are typically covered with an optically transparent sterile drape. The laser can conveniently be activated by means of a computer command or by foot pedal. Such laser/camera devices are commercially available. Laser/camera devices suitable for intra-operative imaging are commercially available. In preferred embodiments, the laser/camera device is a SPY® Intra-operative Imaging System, a HELIOS® Imaging System, or a LUNA® Imaging System (all by Novadaq Technologies, Inc., Mississauga, Ontario, Canada).

In some embodiments, an instrument having an optical configuration similar to a fluorescence microscope may be used, in which a dichroic mirror is used to split the paths of the illumination (the excitation light). The excitation light reflects off the surface of the dichroic mirror into the objective, while the fluorescence emission passes through the dichroic mirror to the eyepiece or is converted into a signal to be presented on a screen. The instrument may further have an excitation filter or an emission filter, or both, to select the wavelengths appropriate for each function. Conveniently, the filters are interference filters, which block transmission of frequencies out of their bandpass.

For immediate observation, ICG is administered intravenously and as the dye passes through the vessels, the excitation light (for ICG, the excitation peak is 805 nm) causes the dye to fluoresce, emitting light with a peak emission at 830 nm. For visualizing torsion, the ICG is administered, allowed to accumulate at the area of interest and then is exposed to excitation light as described above. Light emitted by any ICG present in the areas reached by the excitation light is captured using an imaging system. Typically, the capture system is a CCD or CMOS video camera, which feeds the image to a monitor so that the surgeon can visualize the fluorescence of the dye in the testicle or spermatic cord in real time. Optionally, the camera is also attached to a computer and the image is saved, which not only permits documentation of the torsion, but also can be used for training urologic surgeons, nurses, and other medical staff. Typically, the time required for positioning the device is 2 minutes, while the total time that the scrotal area is illuminated with laser light is 30 seconds. Thus, torsion can be rapidly diagnosed or eliminated as a concern.

The methods described herein are suitable for use in mammals. In a preferred embodiments, the mammals for which the techniques can be used are humans.

### Dyes for Visualization and Administration Thereof

As persons of skill are aware, fluorescent dyes have a particular excitation wavelength which causes the dye to fluoresce and emit light of a particular emission wavelength. Persons of skill will appreciate that a considerable literature is available in the art on the characteristics of different dyes, including their excitation wavelength and emission wavelength. This literature is well known, and will not be set forth in detail herein.

The dye is imaged by exciting it with a light that has an excitation wavelength appropriate for the particular dye used. Persons of skill are aware that a variety of dyes exist, and that each dye has an excitation wavelength and an emission wavelength. Some dyes, for example, fluoresce under ultraviolet (“UC”) illumination while others fluoresce under incandescent illumination. There is a large literature on the use of fluorescent dyes and probes in biological assays, such as Dewey, T. G., Ed., Biophysical and Biochemical Aspects of Fluorescence Spectroscopy, Plenum Publishing (1991), Guilbault, G. G., Ed., Practical Fluorescence, Second Edition, Marcel Dekker (1990), Lakowicz, J. R., Ed., Topics in Fluorescence Spectroscopy Techniques (Volume 1, 1991); Principles (Volume 2, 1991); Biochemical Applications (Volume 3, 1992); Probe Design and Chemical Sensing (Volume 4, 1994); Nonlinear and Two-Photon Induced Fluorescence (Volume 5, 1997); Protein Fluorescence (Volume 6, 2000); DNA Technology (Volume 7, 2003); Plenum Publishing, Lakowicz, J. R., Principles of Fluorescence Spectroscopy, Second Edition, Plenum Publishing (1999), and W. T. Mason, ed., Fluorescent and Luminescent Probes for Biological Activity. A Practical Guide to Technology for Quantitative Real-Time Analysis, Academic Press (Second Ed., 1999).

Preferably, the dye selected is one that has low toxicity and has excitation and emission peaks within the “optical window” of tissue, where absorption due to endogenous chromophores is low. Near infrared light, for example, can penetrate tissue to a depth of several millimeters to a few centimeters. Preferably, the dyes are near infrared fluorochromes, or “NIRF” that emit light in the near infra red spectrum (referred to herein as “near infrared dyes”). In some embodiments, the dye is a tricarbocyanine dye, and in particularly preferred embodiments, is indocyanine green (“ICG”). ICG is commercially available from, for example, Akorn, Inc. (Buffalo Grove, Ill.), which sells it under the name IC-GREEN™. In other embodiments the dye is selected from fluorescein isothiocyanate, rhodamine, phycoerythrin, phycocyanin, allophycocyanin, o-phthaldehyde, fluorescamine, Rose Bengal, trypan blue, and fluoro-gold. The dyes may be mixed or combined. In some embodiments, dye analogs may be used. A “dye analog” is a dye that has been chemically modified, but still retains its ability to fluoresce when exposed to radiant energy of an appropriate wavelength. ICG, Fast Blue and Fluorogold have all been used in mammals with low evidence of neuronal toxicity and are preferred.

ICG is particularly preferred both because it has low toxicity and because it has been approved by the Food and Drug Administration for several diagnostic purposes in humans. Further, its absorption (excitation) and emission peaks (805 and 835 nm, respectively) lie within the “optical window” of tissue. After intravenous injection, ICG is bound within 1 to 2 seconds, mainly to globulins (1-lipoproteins), and remains intravascular, with normal vascular permeability. ICG is not metabolized in the body and is excreted exclusively by the liver, with a plasma half-life of 3 to 4 minutes. It is not reabsorbed from the intestine and does not undergo enterohepatic recirculation. The recommended dose for ICG video angiography is 0.2 to 0.5 mg/kg; the maximum daily dose should not exceed 5 mg/kg.

For visualizing the testicle and spermatic cord intraoperatively, the surgical field, or the portion of the surgical field in which imaging is desired, is illuminated with a light of the excitation wavelength or wavelengths suitable for the dye or dyes used. Ambient light may need to be dimmed to permit the fluorescence to be seen, and observation will typically require magnification. Where the excitation wavelength is outside of the visible range (where, for example, the excitation wavelength is in the ultraviolet or near infrared range), the light source may be designed to permit switching or “toggling” between the excitation wavelength and visible light. This permits the practitioner to note the position of the tissue of interest using the fluorescent property in relation to the rest of the surgical field and surrounding (but non-fluorescent) structures.

As noted in the Introduction, above, in some applications of the invention, the patients will present with sudden scrotal pain and dye will be administered to facilitate diagnosis. In these applications, in which the testicle or spermatic cord are being visualized to determine whether the source of scrotal pain is due to torsion, the dye is typically administered by a bolus injection IV to permit visualizing the blood perfusing the spermatic cord and the testicle. If torsion is found, manual detorsion through the scrotum may be attempted. If not, the patient will typically be taken into surgery as soon as a urologist and an anesthetist or anesthesiologist is available. The patient is then placed under anesthesia and surgery commenced promptly. Typically, the dye administered as part of the diagnostic procedure will remain in the circulation long enough to permit visualization of reperfusion of the testicle once the torsion is relieved during the subsequent surgery. Circulating levels of ICG decline following bolus injections, however, so the concentration of the dye in blood perfusing the testicle is expected to diminish over time. Accordingly, if the time for surgery is expected to be long, or if the practitioner desires to increase the intensity of fluorescence during the procedure, a continuous intravenous infusion or a rapid drip can be used to keep blood levels relatively constant during the operation. Alternatively, a second bolus can be administered. As explained below, however, if the testicle is “borderline,” the administration of a second bolus is preferably used in conjunction with determining whether the borderline testicle can be salvaged.

In some cases, the testicle will be borderline, that is, the pre-operative imaging will show that parts of the testicle are perfused and parts are not. In such cases, a determination may have to be made as to whether or not the testicle can be salvaged. In these cases, it will be desirable not to use a continuous infusion or rapid drip, but rather to permit the concentration of dye to diminish before the operation is performed so that fluorescence from the dye administered pre-operatively diminishes. The testicle is then untwisted during the operation and a fresh bolus of dye is administered. If the testicle then fluoresces, blood flow to the testicle has been restored and the testicle is alive; if it does not, the testicle is dead and the practitioner can make the decision to remove it.

In pre-operative transcutaneous localization of an undescended testicle, the dye is administered and ICG in the blood perfusing the testicle is used to image and thereby locate the testicle. This is especially easy in neonates that are the usual subjects in need of such procedures since their skin is relatively transparent, which facilitates visualization of fluorescence from the testicle, and therefore determination of its location.

In some of the other applications of the invention, such as in neonates undergoing a Fowler-Stephens procedure to draw an intra-abdominal testicle into the scrotum, the patient will be scheduled for surgery. In these cases, the dye may be administered either preoperatively or during the operation before the point at which visualization of the testicle is needed. Conveniently, the dye can be administered in the patient's room just before the patient is taken to the operating room. Alternatively, the dye can be administered in the room in which the operation is to be performed immediately before the operation. If the dye is administered pre-operatively, it is of course desirable that the dye is not administered so long before surgery that so much has been removed by the liver that there is not it does not provide adequate visualization of the testicle. For example, the dye can be administered between about 1 minute to about 4 hours before the intended surgery. In some embodiments, the dye is administered about 1 minute to about 3 hours before the intended surgery. In preferred embodiments, the dye is administered about 5 minutes to about 1 hour before the intended surgery. Usually, the administration is by IV.

The maximum daily dosage of ICG for adults is 2 mg/kg. There is no data available describing the signs, symptoms, or laboratory findings accompanying an overdose of ICG. The LD₅₀ after IV administration ranges between 60 and 80 mg/kg in mice, 50 and 70 mg/kg in rats, and 50 to 80 mg/kg in rabbits.

### EXAMPLES

### Example 1

Intraoperative video angiography is performed with a laser-fluorescence imaging device (Novadaq Technologies, Inc., Toronto, Canada) consisting of a near infrared (NIR) laser light source and a NIR-sensitive digital camcorder. For measurements, the unit is positioned 30 to 40 cm from the area of interest. ICG, dissolved in water, is then injected as a bolus. When ICG is used as the imaging dye, NIR light emitted by the laser light source induces ICG fluorescence. The fluorescence is typically imaged by a video camera, with optical filtering to block ambient and laser light so that only ICG fluorescence is captured. Images can be viewed by the surgical team on screen in real time (typically 25 images/sec). Optionally, the images can be stored on the video camera or transferred to a computer or to storage media for later review or training of others.

### Example 2

Two rats were subjected to unilateral interruption of blood flow to the right testis. In rat A, a bulldog clamp was used, in rat B, a mosquito clamp (a smaller clamp designed to cut off blood flow in small blood vessels) was used. ICG was administered by IV to each rat to determine the ability to visualize the degree to which the testicle underwent hypoperfusion or lack of perfusion.

Prior to administration of ICG, rat A was first subjected to near IR and imaged. No testicular fluorescence was evident prior to ICG administration. 0.5 ml of ICG at a concentration of 2.5 mg/ml was then administered by IV. The right spermatic cord was clamped with a bulldog clamp to create vascular obstruction and the testicles were imaged. There was low fluorescence in the right hemiscrotum and intense fluorescence in the left hemiscrotum. Minimal fluorescence in the right hemiscrotum appeared due to the presence of ICG in the subcutaneous and cutaneous vessels of the scrotum; the differential intensity was obvious. Removal of the bulldog clamp from the spermatic cord resulted in restoration of flow to the testicle. Fluoresence of the right testicle was observed. The less intense fluorescence observed after reperfusion is attributed to the fact that circulating levels of ICG decline following bolus injections, so the concentration of the dye in blood perfusing the testicle is expected to diminish over time.

Observation of rat B, the rat subjected to flow interruption with the mosquito clamp, revealed obvious differential perfusion following occlusion with the clamp. The differential perfusion was not reversible due to tissue injury.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of diagnosing occlusion of blood supply through a spermatic cord to a testicle in a subject, comprising:
(a) administering systemically to said subject a dye which fluoresces at an emission wavelength when said dye is contacted with an excitation wavelength;
(b) exposing (i) the spermatic cord, (ii) the testicle, or (iii) both the spermatic cord and the testicle, to a source of illumination comprising said excitation wavelength under conditions such that any fluorescent dye in the spermatic cord, testicle, or both the spermatic cord and testicle fluoresces;
(c) detecting the presence or absence of fluorescence of said dye in said spermatic cord, said testicle, or both said spermatic cord and said testicle, wherein the presence of fluorescence throughout the spermatic cord, or in the testicle, or both, indicates a lack of occlusion of blood supply to said testicle and the absence of fluorescence throughout the spermatic cord, or in the testicle, or both, indicates an occlusion of blood supply to said testicle.

2. A method of claim 1, wherein said exposing of step (b) is of the testicle.

3. A method of claim 1, wherein said exposing of step (b) is of the spermatic cord.

4. A method of claim 1, wherein said dye is administered intravenously.

5. A method of claim 1, wherein said presence or absence of said fluorescence of said spermatic cord or said testicle is visualized on a image display.

6. A method of claim 1, wherein said source of illumination is a laparoscopic instrument.

7. A method of claim 1, wherein said dye is a near infrared dye.

8. A method of claim 1, wherein said dye is a tricarbocyanine dye or an analog thereof.

9. A method of claim 8, wherein the tricarbocyanine dye is indocyanine green.

10. A method of claim 1, wherein the subject is a human.

11. A method of claim 1, wherein said source of illumination exposes said spermatic cord or said testicle or both to said light through the scrotum.

12. A method of claim 1, wherein said detection of the presence or absence of fluorescence is made through the scrotum.

13. A method of claim 1, wherein said diagnosis is made during a surgical operation.

14. A method of claim 1, wherein said dye is administered within 2 hours of said exposure to said source of illumination.

15. A method of claim 1, wherein said dye is administered within 1 hour of said exposure to said source of illumination.

16. A method of claim 1, wherein said dye is administered and said spermatic cord or said testicle is exposed within 5 minutes to said source of illumination.

17. A method of claim 13, wherein said dye is administered between 5 minutes and 1 hour before said surgical operation.

18. A method of determining the location of an undescended testicle in a neonate, comprising:
(a) administering systemically to a neonate with an undescended testicle a dye which fluoresces at an emission wavelength when said dye is contacted with an excitation wavelength;
(b) exposing the lower abdomen of the neonate to a source of illumination comprising said excitation wavelength under conditions such that fluorescent dye in the undescended testicle fluoresces; and
(c) detecting the presence of fluorescence of said dye in said testicle, thereby localizing said testicle.

19. A method of claim 18 wherein said dye is a near infrared dye.

20. A method of claim 19, wherein said dye is a tricarbocyanine dye or an analog thereof.

21. A method of claim 19, wherein the dye is indocyanine green.

22. A method of claim 18, wherein the exposing of step (b) is from outside the abdomen.

23. A method of claim 18, wherein the exposing of step (b) is from an intra-abdominal laparoscopic instrument.

24. A method of determining perfusion of a testicle in a subject following orchidopexy, comprising:
(a) administering systemically to said subject a dye which fluoresces at an emission wavelength when said dye is contacted with an excitation wavelength;
(b) exposing the testicle to a source of illumination comprising said excitation wavelength under conditions such that fluorescent dye in said testicle fluoresces;
(c) observing the presence or absence of fluorescence of said dye in said testicle, wherein the presence of fluorescence indicates perfusion of the testicle and the absence of fluorescence indicates its absence, thereby determining perfusion of the testicle.

25. A method of claim 24, wherein said dye is a near infrared dye.

26. A method of claim 25, wherein said dye is a tricarbocyanine dye or an analog thereof.

27. A method of claim 25, wherein the dye is indocyanine green.

28. A method of determining whether a subject has suffered testicular trauma, comprising:
(a) administering systemically to said subject a dye which fluoresces at an emission wavelength when said dye is contacted with an excitation wavelength;
(b) exposing the testicle to a source of illumination comprising said excitation wavelength under conditions such that fluorescent dye in said testicle fluoresces;
(c) observing whether the dye is present only in said testicle or is extravasated from said testicle, wherein the presence of said extravasation indicates the presence of testicular trauma and the absence of said extravasation indicates the absence of testicular trauma.

29. A method of claim 28, wherein said dye is a near infrared dye.

30. A method of claim 29, wherein said dye is a tricarbocyanine dye or an analog thereof.

31. A method of claim 29, wherein the dye is indocyanine green.

32. A method of claim 28, wherein the exposing of step (b) is from outside the scrotum.

33. A method of claim 28, wherein said exposing or said detecting, or both, is conducted using a laparoscopic instrument or instruments.

34. A surgical forceps for facilitating observing fluorescence in a subject organ, said forceps comprising a first and a second arm joined by a pivot point, first and second finger grips at a proximal end of each of the first and second arms, and first and second jaws on said first and second arms positioned on the distal side of said pivot point from said finger grips, which first and second jaws have an inward aspect and an outside aspect, wherein at least said first jaw has a shield attached to said outward aspect of said jaw which shield is opaque to fluorescent light.

35. A surgical forceps of claim 34, further wherein said second jaw has a shield attached to said outward aspect of said jaw, which shield is opaque to fluorescent light.

36. A surgical forceps of claim 34, wherein said shield is oval in shape except along an edge attached to said jaw.

37. A surgical forceps of claim 34, wherein said shield is semi-circular in shape except along an edge attached to said jaw.

38. A surgical forceps of claim 34, wherein said jaws do not come into contact when said arms are closed.

39. A surgical forceps of claim 34, wherein said arms further comprise a ratcheting mechanism proximal to said finger grips which, when engaged, holds the arms in a closed position.

40. A surgical forceps of claim 34, wherein said jaws are padded.

41. A surgical forceps of claim 34, wherein said fluorescent light is near infrared light.
